(19) United States
(12) Patent Application Publication
(10) Pub. No.: US 2005/0009884 A1
(43) Pub. Date: Jan. 13, 2005

(54) Calcium blockers to treat proliferative retinal diseases

(76) Inventors: Evan B. Dreyer, Upper St. Clair, PA (US); Scott M. Whitcup, Laguna Hills, CA (US); William A. Hare, Tustin, CA (US); Cun Jian Dong, Irvine, CA (US)

Correspondence Address: BRENT A. JOHNSON;ALLERGAN, INC., 2525 Dupont Drive, T2-7H, Irvine, CA, 92612, US

(21) Appl. No.: 10/734,930

(22) Filed: Dec. 12, 2003

Related U.S. Application Data
(63) Continuation of application No. 10/436,902, filed on May 12, 2003.
Application No. 10/436,902 is a continuation of application No. 10/038,215, filed on Jan. 2, 2002, now Pat. No. 6,573,280.
Application No. 10/038,215 is a continuation of application No. 09/445,832, filed on Dec. 13, 1999, now Pat. No. 6,380,261.
(60) Provisional application No. 60/051,962, filed on Jun. 30, 1997.

(51) Int. Cl. A61K 31/455

(52) U.S. Cl. 514/355

(57) ABSTRACT

Glutamate causes migration and proliferation of retinal pigment epithelium and/or glial cells, and glutamate antagonists can prevent, treat or reduce retinal pigment epithelium and/or glial migration and the subsequent development of proliferative vitreoretinopathy. Avoidance or management of proliferative vitreoretinopathy can be achieved by administering to the patient a compound capable of reducing glutamate-induced retinal cell migration in a concentration effective to reduce such migration.

### CROSS REFERENCE TO RELATED APPLICATIONS

This patent application is a continuation-in-part of U.S. patent application Ser. No. 10/436,902, filed on May 12, 2003, which is a continuation of U.S. patent application Ser. No. 10/038,215, filed on Jan. 2, 2002, which is a continuation of U.S. patent application Ser. No. 09/445,832 which was filed on Dec. 13, 1999 as the U.S. National Patent Application of PCT/US98/12414, which was filed on Jun. 15, 1998 and was based on U.S. Provisional Application 60/051,962, which was filed on Jun. 30, 1997 in the name of Dreyer for CALCIUM BLOCKERS TO TREAT PROLIFERATIVE VITREORETINOPATHY. All of the aforementioned patent applications are expressly incorporated by reference herein.

### FIELD OF THE INVENTION

This invention relates to the treatment of diseases related to the proliferation or migration of retinal pigment epithelium and/or glial cells.

### BACKGROUND OF THE INVENTION

Many diseases or conditions which threaten a person's vision are believed to be related to the migration or proliferation of retinal pigment epithelium and/or glial cells. Some examples of such diseases are non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

### BRIEF DESCRIPTION OF THE INVENTION

We have discovered that glutamate causes migration and proliferation of retinal pigment epithelium and/or glial cells. The use of glutamate antagonists to reduce or control retinal pigment epithelium and/or glial migration and the subsequent development of diseases or conditions is disclosed herein. Disclosed herein is a method of treating a disease or condition wherein migration or proliferation of retinal pigment epithelium or glial cells causes or contributes to the cause of said disease or condition, comprising administering a therapeutically effective amount of a compound which is a glutamate agonist to the patient suffering from said disease or condition.

### DETAILED DESCRIPTION OF THE INVENTION

In relation to the methods of treating disclosed herein, the disease or condition being treated is a disease or condition wherein migration or proliferation of retinal pigment epithelium or glial cells causes or contributes to the cause of said disease or condition. The relationship may be direct or indirect, and the migration or proliferation retinal pigment epithelium or glial cells may be a root cause of said disease or condition, or may be a symptom of another underlying disease or condition. While not intending to limit the scope of the invention in any way, the following are examples of the types of diseases or conditions treated by the disclosed method: non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

In one method, disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, branch retinal vein occlusion, anterior ischemic optic neuropathy, non-retinopathy diabetic retinal dysfunction, and retinitis pigmentosa.

In another embodiment the disease or condition is not proliferative vitreoretinopathy.

In another method, the disease is proliferative diabetic retinopathy.

While not desiring to be bound to any specific theory, we conclude that one or more of the several types of calcium-permeable CNS ion channels mentioned below can be involved in controlling such migration, including: a) the various aspects of the NMDA (N-methyl-D-aspartate) receptor channel complex; b) the voltage-dependent Ca.sup.2+ channels; and c) other channels directly coupled to glutamate (or excitatory amino acid) receptors. Such channels are reviewed in: Sommer, B. and Seeburg, P. H. “Glutamate receptor channels: novel properties and new clones” Trends Pharmacological Sciences 13:291-296 (1992); Nakanishi, S., “Molecular Diversity of glutamate receptors and implications for brain function”, Science 248:597-603 (1992).

The compound may be one of the so-called NMDA antagonists—i.e., it reduces neuronal damage mediated by the NMDA receptor complex. Alternatively, the compound antagonizes neuronal damage mediated by the voltage-dependent calcium channel. Other useful compounds are those which limit release of glutamate from cells or reduce the intracellular neurotoxic consequences of glutamate interaction with cell membrane glutamate receptors. Preferably, the compound crosses the blood-retinal barrier.

Particularly preferred compounds are antagonists of the NMDA receptor-channel complex. The term “NMDA receptor antagonists” includes several sub-types of NMDA antagonists including: a) channel blockers—i.e., antagonists that operate uncompetitively to block the NMDA receptor channel; b) receptor antagonists—antagonists that compete with NMDA to act at the NMDA binding site; c) agents acting at either the glycine co-agonist site or any of several modulation sites such as the zinc site, the magnesium site, the redox modulatory site, or the polyamine site; d) agents which inhibit the downstream effects of NMDA receptor stimulation, such as agents that inhibit activation of protein kinase C activation by NMDA stimulation, antioxidants, and agents that decrease phosphatidylinositol metabolism.

Other compounds that are useful in the invention include voltage-dependent calcium channel antagonists, e.g. those which exert a substantial direct effect on glutamate toxicity mediated by the L-type voltage dependent Ca.sup.++ channel in that they produce a statistically significant result in experiments measuring glutamate induced effects by the general method described in Karschian and Lipton, J. Physiol.418:379-396 (1989) or by other techniques for measuring antagonism of the L-type Ca.sup.++ channel known to those in the art. (We contrast the direct effect so measured with the secondary effects of excitoxicity mediated by other channels, which in turn causes flow through the voltage dependent Ca.sup.++ channels.) Particular candidate compounds include Class I voltage dependent Ca.sup.++ channel antagonists, e.g., phenylalkylamines.

Preferably, the compounds used cross the blood-retina barrier and can be administered chronically. Other useful agents act as antagonists of non-NMDA receptors (glutamate receptor types other than the NMDA receptor complex discussed above), and include agents which block inotropic glutamate receptors or interact with metabotropic glutamate receptors (Nakanishi, supra). Still other agents act to limit (reduce) release of glutamate from cells, thereby acting upstream from the glutamate receptors in the excitatory neurotoxicity process. Still other agents may act by blocking downstream effects of glutamate receptor stimulation, e.g., the intracellular consequences of glutamate interaction with a cell membrane glutamate receptor, such as agents (like dantrolene) that block the rise in intracellular calcium following stimulation of membrane glutamate receptors.

The most preferred compounds are those capable of crossing the blood-retinal barrier; these compounds may be administered orally, intravenously, or topically and cross intervening barriers including the blood-retina barrier to reach the retinal ganglion cells. Compounds that do not freely cross the blood-retina barrier are less preferred; these compounds may be administered intravitreally to the retina. In the case of compounds that have an intermediate ability to cross the blood-retina barrier, the mode of administration will depend on the dosage required and other factors.

Among the preferred compounds are amantadine derivatives (e.g., memantine, amantadine, and rimantadine), nitroglycerin, dextorphan, dextromethorphan, and CGS-19755. See generally, the compounds listed in Table 2.

The invention is useful for the reduction or prevention (including prophylactic treatment) of damage as a result of proliferative vitreoretinopathy.

In view of our discovery that glutamate is associated with proliferative vitreoretinopathy, the invention features antagonists having certain specific characteristics: the ability to cross the blood-retina barrier; and the ability to be administered chronically. Within those guidelines, any suitable antagonist of the glutamate induced excitotoxicity may be used in accordance with the invention. As mentioned, in preferred embodiments, N-methyl-D-aspartate (NMDA) subtype of glutamate receptor-channel complex may be used to reduce or prevent proliferative vitreoretinopathy-related injury. Many antagonists of the NMDA receptor have been identified (Watkins et al., Trends in Pharmacological Sci. 11:25, 1990, hereby incorporated by reference). There are several recognized sub-types of NMDA receptor including: a) channel blockers—i.e., antagonists that operate non-competitively to block the NMDA receptor channel; b) receptor antagonists—antagonists that compete with NMDA, acting at the NMDA binding site; c) agents acting at either the glycine co-agonist site or any of several modulation sites such as the zinc site, the magnesium site, the redox modulatory site, or the polyamine site; d) agents which inhibit the downstream effects of NMDA receptor stimulation such as agents that inhibit activation of protein kinase C activation by NMDA stimulation, antioxidants, and agents that decrease phosphatidylinositol metabolism.

Other compounds that are useful in this invention include non-NMDA receptor antagonists, such as agents which block other types of inotropic glutamate receptors or interact with metabotropic glutamate receptors; voltage-dependent calcium channel antagonists (against L, N, T, and P type channels) (Bean, B. P. Annu. Rev. Physiol. 51:367-384 (1989); Hess, P. Annu. Rev. Neurosci. 13:337-356 (1990)), and are described in greater detail below; and agents which act to decrease the release of glutamate, thereby acting upstream in the excitatory neurotoxicity process.

Table 1, below, lists various suitable NMDA and non-NMDA receptors which do not operate via the voltage-dependent Ca.sup.++ ion channel. Tables 2-4 list antagonists of the voltage dependent Ca.sup.++ channel, which can be used by themselves in connection with the first aspect of the invention, and which can also be used in combination with other antagonists in the second aspect of the invention.

| NMDA Antagonists | | NMDA Antagonists | | NMDA Antagonists | |
|---|---|---|---|---|---|
| 1. | Competitive | 2. | Channel | 3. | Antagonists at |
| | NMDA | | Blockers | | Glycine Site |
| | Antagonists | | (Un-Competitive | | of the NMDA |
| | (act at agonist | | NMDA | | Receptor |
| | binding site) | | Antagonists) | | |
| | CGS-19755 | | MK-801 | | Kyourenate, 7- |
| | (CIBA- | | (Dizocilpine) | | chloro- |
| | GEIGY) | | and other | | kyourenate, |
| | and other | | derivatives | | 5,7-chloro- |
| | piperdine | | of dibenzyocycloheptene | | kyourenate, |
| | derivatives, | | (Merck) | | thio- |
| | D-2-amino-5- | | | | derivatives, |
| | phosphovalerate, | | | | and other |
| | D-2-amino-7- | | | | derivatives. |
| | phosphonoheptanoate | | | | (Merck) |
| | (AP7) | | | | |
| | CPP {[3-(2- | | Sigma receptor | | Indole-2- |
| | carboxy- | | ligands, e.g. | | carboxylic acid |
| | piperazin-4-y- | | Dextrorphan, | | |
| | propyl-1-phosphonic | | dextromethorphan | | |
| | acid]} | | and morphinan | | |
| | | | derivatives | | |
| | | | (Hoffman La | | |
| | | | Roche) such | | |
| | | | as caramiphen | | |
| | | | and | | |
| | | | timeazole | | |
| | | | (which | | |
| | | | also block | | |
| | | | calcium | | |
| | | | channels) | | |
| | LY27614, | | Ketamine, | | DNQX |
| | CGP39551, | | Tiletamine and | | |
| | CGP37849, | | other cyclohexanes | | |
| | LY233536 | | | | |
| | O-phosphobornoserine | | Phencyclidine | | Quinoxaline or |
| | | | (PCP) and | | oxidiazole |
| | | | derivatives, and | | derivatives |
| | | | pyrazine | | including CNQX, |
| | | | compounds | | NMQX |
| | MDL100,453 | | Memantadine, | | Glycine partial |
| | | | amantadine, | | agoinst (e.g. |
| | | | rimantadine | | Hoecht-Roussel |
| | | | and | | P-9939) |
| | | | derivatives | | |
| | | | CNS 1102 and | | |
| | | | related bi- and | | |
| | | | tri- substituted | | |
| | | | guanidines) | | |
| | | | Diamines | | |
| | | | Conantokan | | |
| | | | peptide from | | |
| | | | Cocus | | |
| | | | geographus | | |
| | | | Agatoxin-489 | | |
| 4. | Polyamine Site | 5. | Redox Site of | 6. | Other Non- |
| | of NMDA | | NMDA | | Competitive |
| | Receptor | | Receptor | | NMDA |
| | Arcaine and | | Oxidized and | | Antagonists |
| | related biguanidines | | reduced | | Hoechst |
| | and | | glutathione | | 831917189 |
| | biogenic | | PQQ (pyrroloquinoline) | | SKB Carvedilol |
| | polyamines | | Compounds | | |
| | Ifenprodil and | | that generate | | |
| | related drugs | | Nitric Oxide | | |
| | Diethylene- | | (NO) or | | |
| | triamine SL | | other oxidation | | |
| | 82.0715 | | states | | |
| | | | of nitrogen | | |
| | | | monoxide | | |
| | | | (NO+, NO−) | | |
| | | | including those | | |
| | | | listed in the | | |
| | | | box below | | |
| | 1,10-diamino- | | Nitroglycerin | | |
| | decane (and | | and | | |
| | related inverse | | derivative, | | |
| | agonists) | | Sodium Nitro- | | |
| | | | prusside, and | | |
| | | | other NO | | |
| | | | generating | | |
| | | | listed on p. 5 | | |
| | | | of this table | | |
| | | | Nitric oxide | | |
| | | | sythase (NOS) | | |
| | | | Inhibitors: | | |
| | | | Arginine | | |
| | | | analogs | | |
| | | | including N- | | |
| | | | mono-methyl- | | |
| | | | L-argine | | |
| | | | (NMA): | | |
| | | | N-amino-L- | | |
| | | | arginine | | |
| | | | (NAA); | | |
| | | | N-nitro-L- | | |
| | | | (NNA); | | |
| | | | N-nitro-L- | | |
| | | | arginine methyl | | |
| | | | ester; N-imino- | | |
| | | | ethyl-L- | | |
| | | | ornithine | | |
| | | | Flavin | | |
| | | | Inhibitors: | | |
| | | | diphenyl- | | |
| | | | iodinum; | | |
| | | | Calmodulin | | |
| | | | inhibitors, | | |
| | | | trifluoperizine | | |
| | | | Calcineurin | | |
| | | | Inhibitors, e.g., | | |
| | | | FK-506 | | |
| | | | (inhibits | | |
| | | | calcineurin | | |
| | | | and thus NOS | | |
| | | | diphosphorylase) | | |
| Inhibitors | | Inhibitors | | | |
| of Downstream | | of Downstream | | Non-NMDA | |
| Effects of NMDA | | Effects of NMDA | | Receptor Antagonists | |
| 7. | Agents to | 8. | Downstream | 9A. | Non-NMDA |
| | inhibit protein | | effects from | | antagonists |
| | kinase C | | Receptor | | (Competitive) |
| | activation by | | Activation | | |
| | NMDA stimulation | | | | |
| | (involved in | | | | |
| | NMDA | | | | |
| | toxicity) | | | | |
| | MDL 27.266 | 8a. | To decrease | | CNQX, NBQX, |
| | (Merrill Dow) | | phopshatidylinositol | | YM900, DNQX, |
| | and triazole- | | metabolism | | PD 140532 |
| | one derivatives | | kappa opioid | | AMOA (2-amino- |
| | Monosialo- | | receptor | | 3[3-9carboxy- |
| | gangliosides | | agonist: | | methoxyl-5-methoxylisox- |
| | (eg GM1 | | U50488 | | azol-4-yl] |
| | of Fidia Corp.) | | (Upjohn) | | propionate) |
| | and other ganglioside | | and dynorphan | | |
| | derivatives | | | | |
| | LIGA20, | | | | |
| | LIGA4 | | | | |
| | (may also | | | | |
| | effect calcium | | | | |
| | extrusion | | | | |
| | via calcium | | | | |
| | ATPase) | | | | |
| | | | kappa opioid | | 2-phosphophonoethyl |
| | | | receptor | | phenylalamine |
| | | | agonist: | | |
| | | | PD117302, | | derivatives, i.e. |
| | | | CI-977 | | 5-ethyl, 5-methyl, |
| | | | | | 5-trifluoromethyl |
| | | 8b. | To decrease | | |
| | | | hydrogen | | |
| | | | peroxide and | | |
| | | | free radical | | |
| | | | injury, eg | | |
| | | | antioxidants | | |
| | | | 21- | 9B. | Non-NMDA |
| | | | aminosteroid | | Non competitive |
| | | | (lazaroids) | | antagonists |
| | | | such as | | |
| | | | U74500A, | | |
| | | | U75412E and | | |
| | | | U74006F | | |
| | | | U74389F, | | GYK152466 |
| | | | FLE26749, | | |
| | | | Trolex (water | | |
| | | | soluble alpha | | |
| | | | tocophenol), | | |
| | | | 3,5-dialkoxy-4- | | |
| | | | hydroxy- | | |
| | | | benzylamines | | |
| | | | Compounds | | Evans Blue |
| | | | that generate | | |
| | | | Nitric Oxide | | |
| | | | (NO) or | | |
| | | | other oxidation | | |
| | | | states of | | |
| | | | nitrogen | | |
| | | | monoxide | | |
| | | | (NO+, NO−) | | |
| | | | including | | |
| | | | those listed in | | |
| | | | the box below | | |
| | | | Nitroglycerin | | |
| | | | and | | |
| | | | derivatives, | | |
| | | | Sodium Nitro- | | |
| | | | prusside, and | | |
| | | | other NO | | |
| | | | generating | | |
| | | | listed on p. 5 | | |
| | | | of this | | |
| | | | table | | |
| | | | Nitric oxide | | |
| | | | synthase (NOS) | | |
| | | | Inhibitors: | | |
| | | | Arginine | | |
| | | | analogs including | | |
| | | | N- | | |
| | | | mono-methyl- | | |
| | | | L-arginine | | |
| | | | (NMA); N- | | |
| | | | amino-L- | | |
| | | | arginine | | |
| | | | (NAA); N- | | |
| | | | nitro-L- | | |
| | | | arginine | | |
| | | | (NNA); N- | | |
| | | | nitro-L- | | |
| | | | arginine methyl | | |
| | | | ester, N- | | |
| | | | iminoethyl-L- | | |
| | | | ornithine | | |
| Agents Active at | | | | Drugs to decrease | |
| metabotropic | | | | intracellular calcium | |
| Glutamate | | Decrease | | following glutamate | |
| Receptors | | glutamate release | | receptor stimulation | |
| 10a. | Blockers of | 11. | Agents to | 12a. | Agents to |

| | Metabotropic | decrease | | decrease |
|---|---|---|---|---|
| | Glutamate | glutamate | | intracellular |
| | Receptors | release | | calcium release |
| | AP3 (2-amino- | Adenosine, and | | Dantrolene |
| | 3-phosphono- | derivatives, | | (sodium |
| | prionic acid) | e.g. cyclo- | | dantrium); |
| | | hexyladenosine | | Ryanodine (or |
| | | | | ryanodine + caffiene) |
| 10b. | Agonists of | CNS1145 | 12b. | Agents inhibiting |

| | | |
|---|---|---|
| Metabotropic | | intracellular |
| Glutamate | | Calcium- |
| Receptors | | ATPase |
| (1S,3R)-1- | Conopeptides: | Thapsigargin, |
| Amino-cyclo- | SNX-111, | cyclopiazonic |
| pentane-1,3- | SNX-183, | acid, BHQ |
| dicarboxylic | SNX-230 | ([2,5-di-1,4- |
| acid [(1S,3R)- | | (tert butyl)- |
| ACPD], | | benzohydro- |
| commonly ref | | quinone; |
| as \grave{ }trans\grave{ }- | | 2,5-di-(tert |
| ACPD | | butyl)-1,4 |
| | | benzohydro- |
| | | quinone]) |
| | Omega-Age- | |
| | IVA, toxin | |
| | from venom | |
| | of funnel | |
| | web spider | |
| | Compounds | |
| | that generate | |
| | Nitric Oxide | |
| | (NO) or other | |
| | oxidation states | |
| | of nitrogen | |
| | monoxide | |
| | (NO+, NO−) | |
| | including | |
| | those listed | |
| | in the box | |
| | below | |
| | Nitroglycerin | |
| | and | |
| | derivatives, | |
| | Sodium Nitro- | |
| | prusside, and | |
| | other NO | |
| | generating | |
| | listed on p. 5 | |
| | of this table | |
| | Nitric oxide | |
| | synthase (NOS) | |
| | Inhibitors: | |
| | Arginine | |
| | analogs | |
| | including N- | |
| | mono-methyl- | |
| | L-arginine | |
| | (NMA); | |
| | N-amino-L- | |
| | arginine (NAA) | |
| | N-nitro-L- | |
| | arginine | |
| | (NNA); | |
| | N-nitro-L- | |
| | arginine methyl | |
| | ester; | |
| | N-iminoethyl- | |
| | L-ornithine | |
| | Additional NO- | |
| | generating | |
| | compounds | |
| | Isosorbide | |
| | dinitrate | |
| | (isordil) | |
| | S-nitrosocaptopril | |
| | (SnoCap) | |
| | Serum albumin | |
| | coupled to | |
| | nitric oxide | |
| | (SA-NO) | |
| | Cathepsin | |
| | coupled to | |
| | nitric oxide | |
| | (cathepsin-NO) | |
| | Tissue | |
| | plasminogen | |
| | activator | |
| | coupled to | |
| | NO (TPA-NO) | |
| | SIN-1 (also | |
| | known as SIN1 | |
| | or molsidomine) | |
| | Ion-nitrosyl | |
| | complexes | |
| | (e.g., | |
| | nitrosyl-iron | |
| | complexes, | |
| | with iron in the | |
| | Fe2+ state) | |
| | Nicorandil | |

TABLE 2
Antagonists of the Voltage Dependent Calcium Channels
(N, L, T, P and other types)

dihydropyridines
(e.g., nimodipine)
phenylalkylamines
(e.g., verapamil, (S)-emopamil, D-600, D-888)
benzothiazepines
(e.g., diltiazem and others)
bepridil and related drugs
diphenylbutylpiperdines
diphenylpiperazines
(e.g., flunarizine/cinnarizine series)
HOE 166 and related drugs
fluspirilene and related drugs
toxins and natural compounds
(e.g., snail toxins —
.omega.conotoxin GVIA and GVIIA, maitotoxin,
taicatoxin, tetrandine, hololena toxin, plectreurys
toxin, funnel-web spider venom and its toxin fraction,
agatoxins including .omega.-agatoxin IIIA and .omega.-
agatoxin IVA.

TABLE 2
Antagonists of the Voltage Dependent Calcium Channels
(N, L, T, P and other types)

dihydropyridines
(e.g., nimodipine)
phenylalkylamines
(e.g., verapamil, (S)-emopamil, D-600, D-888)
benzothiazepines
(e.g., diltiazem and others)
bepridil and related drugs
diphenylbutylpiperdines
diphenylpiperazines
(e.g., flunarizine/cinnarizine series)
HOE 166 and related drugs
fluspirilene and related drugs
toxins and natural compounds
(e.g., snail toxins -
.omega.conotoxin GVIA and GVIIA, maitotoxin,
taicatoxin, tetrandine, hololena toxin, plectreurys
toxin, funnel-web spider venom and its toxin fraction,
agatoxins including .omega.-agatoxin IIIA and .omega.-
agatoxin IVA.

TABLE 4
OTHER CALCIUM CHANNEL ANTAGONISTS

| | |
|---|---|
| diclofurime | D-600 |
| pimozide | D-888 |
| prenylamine | Smith Kline 9512 |
| fendiline | ranolzine |
| perhexiline | lidoflazine |
| mioflazine | CERM-11956 |
| flunarizine/ | R-58735 |
| cinnarizine series | R-56865 |
| verapamil | amiloride |
| dilfiazine | phenytoin |
| dipropervine | thioridazine |
| (S)-emopamil | tricyclic antidepressents |

In Vitro Assay

An antagonist may be tested for utility in the method of the invention by monitoring its effect on proliferative retinopathy as follows.

Cultured fibroblasts will be injected into the vitreous of the rabbit eye. After two weeks, the degree of vitreopathy can be assessed histologically. At the time of the initial insult, the animals will be treated with the compound under consideration.

Such models are well known. A few examples (hereby incorporated by reference) included Kiumura et al. Human Gene Therapy, 7:799-808 (1996); Sakamoto et al., Ophthalmology 102:1417-1421 (1995); Handa et al. Experimental Eye Research 62:689-696 (1996); Berger et al. 37:2318-1325 (1996); de Souza et al. Ophthalmologica 209:212-216 (1995); Nakagawa et al. Ophthalmology & Visual Science 36:2388-2395 (1995); Steinhorst et al. Archive for Clinical & Experimental Ophthalmology 232:347-354 (1994).

Use

An effective receptor antagonist will cause a decrease in proliferative vitreoretinopathy. As described above, the preferred compounds which cross the blood-retinal barriers are preferably administered topically or orally in known, physiologically acceptable vehicles including tablets, liquid excipients and suspensions. Those skilled in the art will appreciate how to formulate acceptable therapeutics.

Antagonists may be compounded into a pharmaceutical preparation, using pharmaceutical compounds well-known in the art; the exact formulation and dosage of the antagonist compound depends upon the route of administration. Generally, the effective daily dose of the antagonists will range from 0.01 to 1000 mg/kg.

Other Embodiments

Other embodiments are within the following claims. In the method of the invention, a useful compound may be administered by any means that allows the compound access to the retina. The compounds useful in the method include antagonists of excitatory amino acid receptors (both NMDA and non-NMDA subtypes) that act to reduce retinal cell migration or proliferation or reduce binding of glutamate to the NMDA receptor. The antagonists can act at a modulatory site or a co-agonist site or by blocking the chain of events initiated by receptor activation.

1. A method of treating a disease or condition wherein migration or proliferation of retinal pigment epithelium or glial cells causes or contributes to the cause of said disease or condition, comprising administering a therapeutically effective amount of memantine to the patient suffering from said disease or condition.

2. The method of claim 1 wherein said disease or condition is not proliferative vitreoretinopathy.

3. The method of claim 1 wherein said disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

4. The method of claim 1 wherein said disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, branch retinal vein occlusion, anterior ischemic optic neuropathy, non-retinopathy diabetic retinal dysfunction, and retinitis pigmentosa.

5. The method of claim 1 wherein said disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

6. The method of claim 1 wherein said disease is proliferative diabetic retinopathy.

7. The method of claim 1 wherein the memantine is administered orally.

8. The method of claim 1 wherein the memantine is administered topically.

9. The method of claim 1 wherein the memantine is administered to the eye or the surrounding tissue via implant or injection.

10. The method of claim 1 wherein the memantine is administered chronically.

11. A method of treating a disease or condition, comprising administering a therapeutically effective amount of memantine to a mammal suffering from said disease or condition, wherein said disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

12. The method of claim 11 wherein said disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, branch retinal vein occlusion, anterior ischemic optic neuropathy, non-retinopathy diabetic retinal dysfunction, and retinitis pigmentosa.

13. The method of claim 11 wherein said disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

14. The method of claim 11 wherein said disease is proliferative diabetic retinopathy.

15. The method of claim 11 wherein the memantine is administered orally.

16. The method of claim 11 wherein the memantine is administered topically.

17. The method of claim 11 wherein the memantine is administered to the eye or the surrounding tissue via implant or injection.

18. The method of claim 11 wherein the memantine is administered chronically.
